(19) United States
(12) Patent Application Publication
(10) Pub. No.: US 2011/0200618 A1
(43) Pub. Date: Aug. 18, 2011

(54) ERBB4 INHIBITORS AND USES THEREOF IN TREATMENT OF NEUROPSYCHIATRIC DISORDERS

(76) Inventor: Chang-Gyu Hahn, Bryn Mawr, PA (US)

(21) Appl. No.: 12/867,650

(22) PCT Filed: Feb. 17, 2009
(86) PCT No.: PCT/US2009/034311

§ 371 Date: Apr. 4, 2011

Related U.S. Application Data
(60) Provisional application No. 61/064,072, filed on Feb. 14, 2008.

(51) Int. Cl. A61K 39/395; A61P 25/24; A61P 25/22; A61P 25/18; A61K 33/00; C12Q 1/48; A61K 31/517; A61K 31/277; A61K 31/35

(52) U.S. Cl. 424/172.1; 424/722; 435/7.1; 514/266.4; 514/519; 514/453

(57) ABSTRACT

This invention relates to treatment of neuro-psychiatricdisorders. Specifically, the invention relates to the use of small molecule agent specific against erbB4, erbB3 or their combination, or fragments thereof in inhibiting the attachment of erbB4, erbB3 or their combination to NRG1.

### FIELD OF INVENTION

This invention is directed to treatment, modulation, suppression, inhibition or amelioration of neuro-psychiatric disorders symptoms and indications. Specifically, the invention is directed to the use of small molecules antagonists of erbB4 in inhibiting the attachment of erbB4 to NRG1 so as to modulate N-methyl-Daspartate (NMDA) receptor signaling, thereby treating neuro-psychiatric disorder.

### BACKGROUND OF THE INVENTION

Schizophrenia, affects approximately 2 million Americans. At any particular time, about 20% of the hospital beds in the U.S. are occupied by schizophrenic patients. The illness usually develops between adolescence and age 30 and is characterized by positive symptoms (delusions or hallucinations), negative symptoms (blunted emotions and lack of interest) and disorganized symptoms (confused thinking and speech or disorganized behavior and perception). Additionally, cognitive deficits are also frequently observed, particularly in elderly schizophrenia patients (Purohit et al., 1993, Biol. Psychiatry 33(4):255 260). For some patients, the disorder is lifelong, while others may have periodic episodes of psychosis.

Several putative schizophrenia susceptibility genes have been identified. Genomewide linkage studies and metaanalyses of linkage scans have highlighted chromosome 8p as a susceptibility locus. Extensive fine-mapping of the 8p locus, haplotype-association analysis, and linkage disequilibrium (LD) tests subsequently implicated neuregulin 1 (NRG1), a gene with pleotropic roles in neurodevelopment and plasticity.

NRG1-mediated erbB signaling has important roles in neural and glial development, as well as in the regulation of neurotransmitter receptors thought to be involved in the pathophysiology of schizophrenia. ErbB4 is of particular interest in relation to the pathophysiology of schizophrenia because erbB4 signaling can modulate neurobiological processes often disturbed in the disorder: neuronal migration, the biology of GABAergic interneurons and NMDA receptor (NMDAR) transmission. Attempts have been made to examin the expression of NRG1 mRNAs in postmortem prefrontal cortex of schizophrenic subjects, with variable results: an overall increase, an increase in type I mRNA or subtle changes in the ratio of type II/type I or type II/type III mRNA (R. Navon et al., Abstr. XIIth World Congr. Psychiatr. Genet. P8.20, 2004; J. Law et al., Soc. Neurosci. Abstr. 109.7, 2004; and ref. 17, respectively). To date, however, no specific role for NRG1 has been established in schizophrenia.

Therefore a need still remains in the art for an effective, and long lasting treatment of the symptoms of schizophrenia, without serious side effects, with future treatment regimes and drug development efforts requiring a more sophisticated approach focused on genetic causes and their modulation.

### SUMMARY OF THE INVENTION

In one embodiment, the invention provides a method of treating, inhibiting or suppressing, or ameliorating symptoms associated with a neuropsychiatric disorder in a subject, inhibiting or suppressing a neuropsychiatric disorder in a subject, or ameliorating symptoms associated with a neuropsychiatric disorder in a subject, comprising the step of administering to the subject a small molecule agent, or an antibody or its fragment in an amount sufficient to inhibit the attachment of erbB4, erbB3 or their combination to NRG1.

In another embodiment, the invention provides a composition for treating a neuropsychiatric disorder in a subject comprising a small molecule agent, or an antibody or its fragment capable of inhibiting the attachment of erbB4, erbB3 or their combination to NRG1.

In one embodiment, the invention provides a method of inhibiting neuregulinl (NRG1) activation of protein kinase B (AKT), extracellular signal-regulated kinase (ERK) or both, comprising the step of contacting a brain cell with a composition comprising a small molecule agent capable of inhibiting the attachement of erbB4 to NRG1.

In another embodiment, the invention provides a method of screening for an agent for the treatment of neuropsychiatric disorders, comprsing the steps of obtaining a biological sample from a subject or pool of subjects diagnosed with having a neuropsychiatric disorder; incubating the biological sample with a candidate agent for a predetermined period; following incubation, contacting the biological sample with neuregulinl; and analyzing whether the candidate agent is capable of inhibiting the attachement of erbB4 to NRG1, wherein a candidate agent capable of inhibiting the attachement of erbB4 to NRG1 is useful for the treatment of psychiatric disorder.

In one embodiment, the invention provides a method of inhibiting NMDAR 2B phosphorylation in a cell, comprising the step of contacting the cell with a composition comprising an agent capable of inhibiting erbB4-NRG1 attachment.

In another embodiment, the invention provides a method of inhibiting NMDAR1 association with phosphatidyl inositole phospholipase C-γ (PIPLCγ) in a cell, comprising the step of contacting the cell with a small molecule agent capable of inhibiting erbB4-NRG1 attachment.

In one embodiment, the invention provides a method of treating, inhibiting or suppressing or ameliorating symptoms associated with schizophrenia in a subject, comprising the step of administering to the subject a small molecule agent, in an amount sufficient to inhibit the attachment of erbB4, to NRG1, PSD-95 or both, thereby treating, inhibiting or suppressing or ameliorating symptoms associated with schizophrenia.

In another embodiment, the invention provides a method of treating, inhibiting or suppressing or ameliorating symptoms associated with bipolar disorder in a subject, comprising the step of administering to the subject a small molecule agent, in an amount sufficient to inhibit the attachment of erbB4, to NRG1, PSD-95 or both, thereby treating, inhibiting or suppressing or ameliorating symptoms associated with bipolar disorder.

In one embodiment, the agent capable of inhibiting NRG1-erbB4 attachement is AG1487, CI1103, AG879, PD158780, PD168393, GSK1495829A, GSK1521232A, or their combination.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows NRG1—erbB activation in hOE cultures. hOE cultures were pre-incubated with inhibitors for 20 min, were then added with 100 ng/ml of NRG1 for 10 min at 37° C. Protein extracts were analyzed for the expression of p-ERK and glyeraldehyde 3-dehydrogenase (GAPDH). U, 10 μM of U0126: W, 10 nM of wortmannin: K, 100 nM K-252, 2.5 μM of AG1478 and 1 μM of AG879;

FIG. 2 shows FIG. 2. NRG1 stimulation of human post-mortem PFC. 100 mg of post-mortem brain tissues of a healthy control subject were incubated with or without 100 ng/ml of NRG1 in the presence or absence of 2.5 μM AG1478. Protein extracts were analyzed for p-ERK and ERK;

FIG. 3 shows hOE cultures were pre-incubated with inhibitors for 20 min, were then added with 100 ng/ml of NRG1 for 10 min at 37° C. Protein extracts were analyzed for the expression of p-ERK and glyceraldehyde 3-dehydrogenase (GAPDH). (A) A representative western blotting (B) p-ERK intensities were normalized with respect to GAPDH, which were then used to calculate percent inhibition. Blue: pAKT/AKT, Red, pERK 44 kD/ERK and Yellow, pERK 42 kD/ERK;

FIG. 4 shows 100 mg of post-mortem brain tissues of a healthy control subject were pre-incubated with varied concentrations of GSK1495829A for 15 min, then were incubated with 100 ng/ml of NRG1. Protein extracts were analyzed for the expression of ERK, AKT and GSK and their phosphorylated counterparts;

FIG. 5 shows 100 mg of post-mortem mouse brain tissues were pre-incubated with varied concentrations of GSK1495829A or AG1467 or vehicle for 60 min, then were incubated with 100 ng/ml of NRG1. Protein extracts were analyzed for the expression of p-ERK;

FIG. 6 shows hOE cultures were pre-incubated with inhibitors for 15 min, were then added with 100 ng/ml of NRG1 for 10 min at 37° C. Protein extracts were analyzed for the expression of p-AKT, p-ERK, and glyceraldehyde 3-dehydrogenase (GAPDH). A representative western blotting for p-AKT. GSK 149529 attenuates NRG1 induced increases in pAKT in a dose depenent manner. GSK 1521123 has similar effects;

FIG. 7 shows hOE cultures were incubated with 100 ng/ml of NRG1 and 100 μM of NMDA and 1 μM of glycine for 15 min at 37° C. Protein extracts were analyzed for pY 1472 NMDAR 2B;

FIG. 8 shows Mouse brain tissues were incubated with 100 ng/ml of NRG1 and 100 μM of NMDA and 1 μM of glycine for 15 min at 37° C. Protein extracts were immunoprecipitated for NMDAR 1 and probed for PIPLCγ;

FIG. 9 shows NRG1 induced erbB4 activation. OVCAR cells were incubated with 100 ng/ml of NRG1 in the presence and absence of GSK1495829A. Protein extracts were immunoprecipitated with antibodies for erbB4 and probed with antibodies for phosphotyrosine;

FIG. 10 shows BE(2)-M17 cells grown in the presence of retinoic acid were incubated with 100 ng/ml of NRG1 and 100 μM of NMDA and 1 μM of glycine for 15 min at 37° C. in the presence and absence of GSK1495829A. Protein extracts were immunoblotted for pSRC, pPyK, pY1472 but not pCAMKII; and

FIG. 11 shows Human post-mortem brain tissues were incubated with 100 ng/ml of NRG1 and 100 μM of NMDA and 1 μM of glycine for 15 min at 37° C. Protein extracts were immunoprecipitated for NMDAR 1 and probed for PIPLCγ.

### DETAILED DESCRIPTION OF THE INVENTION

This invention relates in one embodiment to treatment of neuro-psychiatricdisorders. In another embodiment, the invention provides the use of small molecule agent specific against erbB4, erbB3 or their combination, or fragments thereof in inhibiting the attachment of erbB4, erbB3 or their combination to NRG1.

In one embodiment, Neuregulin-1 (NRG1) family consists of structurally related proteins containing an epidermal growth factor (EGF)-like domain that specifically activate receptor tyrosine kinases of the erbB family: erbB2, erbB3 and erbB4. These isoforms are divided into three classic groups: type I (previously known as acetylcholine receptor inducing activity, heregulin, or neu differentiation factor), type II (glia growth factor) and type III (cysteine-rich domain containing), which are based on distinct amino termini. Additional NRG1 5′ exons have recently been identified, giving rise putatively to novel NRG1 types IV-VI in the human brain.

In another embodiment, ligand-dependent activation of ErbB receptors results in homo- or heterodimerization, which stimulates receptor trans-phosphorylation on cytoplasmic tyrosine residues, creating binding sites for adaptor or enzymatic proteins. EGF receptor and ErbB4 homodimers are active kinases in the absence of coreceptors and ErbB4 activated in one embodiment, by either homo- or heterodimerization. In one embodiment, ErbB4 expression is necessary to confer on neural progenitor cells the ability to respond to NRG1β1 through migration. In one embodiment, erbB4 receptor contains an extracellular ligand-binding domain of 600-630 amino acids, a single transmembrane α-helix, plus an intracellular domain of ˜600 amino acids that includes the tyrosine kinase and regulatory sequences.

In one embodiment, erbB4 signaling stimulation in the methods and compositions described herein, results in modulation of erbB4-PSD95 binding. In another embodiment, assessing erbB4 signaling in a prefrontal cortex of a schizophrenic subject, comprising the step of stimulating postmortem brain tissues of said subject with an effective amount of Neuregulin-1 (NRG1), thereby enhancing tyrosine phosphorylation of erbB4, further comprises attenuating NMDAR function.

In one embodiment, contacting PFC slices from a subject afflicted with neuropsychiatric disorders with NMDAR stimulantsm causes NMDAR activation, such as increased phosphorylation of subunit 2A of the NMDAR in one embodiment and recruitment of phosphatidyl inositol phospholipase C-γ1 (PIPLC-γ1) by the NMDAR1 subunit in another embodiment in an amount significantly lower than in PFC tissue from subjects not afflicted with neuropsychiatric disorder. In another embodiment, adding NRG1 attenuates NMDAR activation even further.

In one embodiment, provided herein is a method of treating a neuropsychiatric disorder in a subject, comprising the step of administering to the subject an small molecule agent specific against erbB4, erbB3 or their combination, or fragments thereof in an amount sufficient to inhibit the attachment of erbB4, erbB3 or their combination to NRG1, PSD-95 or both. In another embodiment, provided herein is a method of inhibiting or suppressing, or in yet another embodiment, eliminating symptoms associated with a neuropsychiatric disorder in a subject, comprising the step of administering to the subject an small molecule agent specific against erbB4, erbB3 or their combination, or fragments thereof in an amount sufficient to inhibit the attachment of erbB4, erbB3 or their combination to NRG1, PSD-95 or both.

In another embodiment, provided herein is a method of treating a neuropsychiatric disorder in a subject, or, in another embodiment, inhibiting or suppressing a neuropsychiatric disorder in a subject, or, in another embodiment, ameliorating symptoms associated with a neuropsychiatric disorder in a subject, comprising the step of administering to the subject AG1487 in an amount sufficient to inhibit the attachment of erbB4, erbB3 or their combination to NRG1. In one embodiment, the agent administered to the subject in an amount sufficient to inhibit the attachment of erbB4, erbB3 or their combination to NRG1 is CI1103. In one embodiment, the agent administered to the subject in an amount sufficient to inhibit the attachment of erbB4, erbB3 or their combination to NRG1 is AG879. In one embodiment, the agent administered to the subject in an amount sufficient to inhibit the attachment of erbB4, erbB3 or their combination to NRG1 is PD158780. In one embodiment, the agent administered to the subject in an amount sufficient to inhibit the attachment of erbB4, erbB3 or their combination to NRG1 is PD168393, or in one embodiment, the agent administered to the subject in an amount sufficient to inhibit the attachment of erbB4, erbB3 or their combination to NRG1 is their combination.

In one embodiment, erbB4-PSD-95 association is distinctly increased in schizophrenia, with PSD-95 protein levels unaltered, indicating that protein-protein interactions of PSD-95 is important mode of dysregulation in the disease and inhibiting the interaction or binding of erbB4 and PSD95 is effective in controlling the dysregulation of schizophrenia, using the methods described herein.

In one embodiment, schizophrenia is caused by dysregulation of synaptic plasticity in adult subject. ErbB4 receptor is enriched in postsynaptic densities (PSD) and interact with other PSD proteins such PSD-95 (a PDZ domain-containing protein known to aid in receptor scaffolding, interacts primarily with ErbB4 at neuronal synapses where it enhances neuregulin (NRG)-induced kinase activity), NMDA receptor subunit 2C and 2B, Ca2+-activated potassiμM channels, protein kinase C interacting protein (PICK1) and glutamate (AMPA subtype) receptors.

In another embodiment, NRG1 is implicated in susceptibility to bipolar disorder. In another embodiment, subjects with bipolar disorder who experience predominantly mood-incongruent psychotic features show evidence of an influence of susceptibility from NRG1. In one embodiment, NRG1 is responsible for genome-wide linkage in the 8p12 region (the same chromosome where variation at the neuregulin 1 (NRG1) gene influences susceptibility to schizophrenia), to psychosis in bipolar pedigrees.

In one embodiment, stimulation of erbB4 signaling, caused by contact with NRG1 is pathognomonic of schizophrenia, bipolar disorder or their combination and its attenuation is desirable in the treatment of schizophrenia or bipolar disorder in another embodiment, in subjects exhibiting hyperexpression of NRG1, erbB4 or both.

Accordingly, the neuropsychiatric disorders treated using the methods and compositions described herein, is scizophrenia in one embodiment, bipolar disorder in another embodiment, or their combination or variation in another embodiment. In one embodiment, the neuropsychiatric disorders treated, inhibited or suppressed or otherwise whose symptoms are ameliorated using the compositions and methods described herein, are monopolar depression, anxiety and obessessive-complulsive disorder (OCD). In another embodiment, the term “neuropsychiatric disorder” refers to a disease having a pathophysiological component of attenuated NMDA receptor-mediated neurotransmission. These include in one embodiment, Alzheimer's disease, or autism, depression, benign forgetfulness, childhood learning disorders, closed head injury, and attention deficit disorder. Accordingly it is suggested that the methods described herein will be effective to a dgeree in the treatment of those neuropsychiatric diseases where NMDA functioning is attenuated.

In one embodiment, schizophrenia refers to a group of neuropsychiatric disorders characterized by dysfunctions of the thinking process, such as delusions, hallucinations, and extensive withdrawal of the patient's interests from other people. Approximately one percent of the worldwide population is afflicted with schizophrenia, and this disorder is accompanied by high morbidity and mortality rates. The group comprises in another embodiment, schizophrenia, schizophreniform disorder, schizoaffective disorder and psychotic disorder wherein the term “psychotic” refers in one embodiment to delusions, prominent hallucinations, disorganized speech or disorganized or catatonic behavior. In one embodiment, the psychiatric manifestation is characterized by a range of cognitive and emotional dysfunctions that include perception, inferential thinking, language and communication, behavioral monitoring, affect, fluency and productivity of thought and speech, hedonic capacity, volition and drive, and attention. The active-phase symptoms of schizophrenia include delusions, hallucinations, disorganized speech, grossly disorganized behavior, catatonic behavior, and negative symptoms. In one embodiment, these and other similar symptoms are treated and ameliorated using the compositions and methods described herein. In one embodiment, neurocognitive abilities decline with each psychotic episode in subject afflicted with schizophrenia and the methods described herein reduce the frequency of psychotic episodes or in another embodiment, reduce the decline in neurocognitive abilities after each psychoptic episode. In one embodiment, The term “neurocognitive” refers to those mental functions for which physiological indices can be measured and in another embodiment, the term “neurocognitive workload” refers to the level of neural activation associated with mental effort. In one embodiment, the compositions and methods described herein normalizes the post psychotic episode neurocognitive workload in the subject.

“Treating” or “treatment” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term. Therefore, in one embodiment, the invention provides a method of treating schizophrenia or bipolar disorder in another embodiment, in a subject, comprising administering to said subject an agent capable of inhibiting the contact between erbB4, or in another embodiment, erbB3 or both, to NRG1 in one embodiment, or PSD-95 in another embodiment, or both in yet another discrete embodiment. In one embodiment, the agent administered to the subject is GSK1495829A in an amount sufficient to inhibit the attachment of erbB4, erbB3 or their combination to NRG1. In another embodiment, the agent administered to the subject is GSK1521232A in an amount sufficient to inhibit the attachment of erbB4, erbB3 or their combination to NRG1. In another embodiment, the agent administered to the subject is AG1487 in an amount sufficient to inhibit the attachment of erbB4, erbB3 or their combination to NRG1. In one embodiment, the agent administered to the subject in an amount sufficient to inhibit the attachment of erbB4, erbB3 or their combination to NRG1 is CI1103. In one embodiment, the agent administered to the subject in an amount sufficient to inhibit the attachment of erbB4, erbB3 or their combination to NRG1 is AG879. In one embodiment, the agent administered to the subject in an amount sufficient to inhibit the attachment of erbB4, erbB3 or their combination to NRG1 is PD158780. In one embodiment, the agent administered to the subject in an amount sufficient to inhibit the attachment of erbB4, erbB3 or their combination to NRG1 is PD168393, or in one embodiment, the agent administered to the subject in an amount sufficient to inhibit the attachment of erbB4, erbB3 or their combination to NRG1 is their combination.

In another embodiment, provided herein is a composition comprising a therapeutically effective amount of GSK1495829A capable of inhibiting the contact between erbB4, or in another embodiment, erbB3 or both, to NRG1 in one embodiment, or PSD-95 in another embodiment, or both in yet another discrete embodiment. In one embodiment, the compositions provided herein comprise a therapeutically effective amount of GSK1521232A, capable of inhibiting the contact between erbB4, or in another embodiment, erbB3 or both, to NRG1 in one embodiment, or PSD-95 in another embodiment, or both in yet another discrete embodiment. In one embodiment, the compositions provided herein, further comprise one or more additional active pharmaceutical ingredient, effective in the treatment of schizophrenia or bipolar disorder (BPD). In one embodiment, the one or more additional compound is AG1487, or CI1103, AG879, PD158780, PD168393, or their combination in other discrete embodiments of the one or more additional API's used in the compositions described herein.

In one embodiment, the small molecule agent used in the methods and compositions described herein is quinazoline derivatives, where quinazoline is described by the following formula (I):

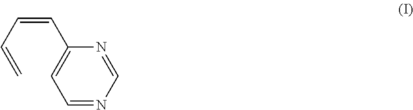

In another embodiment, the quinazoline derivative is 4-(3-Chloroanilino)-6,7-dimethoxyquinazoline (Ag 1478), as set forth in formula (II):

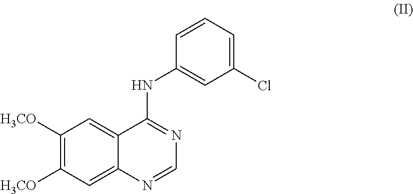

In one embodiment, the quinazoline derivative is 4-anilinoquinazoline (CI 1033; canertinib dihydrochloride) as set forth in formula (III):

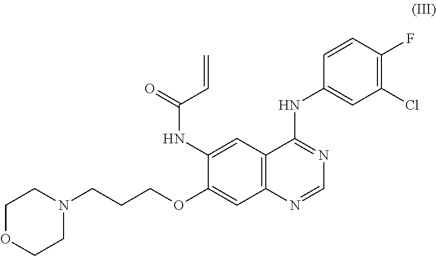

In one embodiment, the small molecule agent used in the methods and compositions described herein is pyrazole derivatives, described by the following formula (IV), so long as these derivatives are effective in the inhibition of attachment of erbB4 to NRG1:

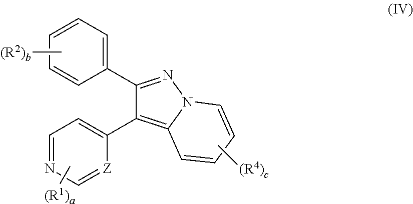

wherein Z is N; a is 1 or 2; b is 1, 2or 3; c is 1, 2 or 3;
each R¹ is independently selected from groups of the formula —(X)_{d}—(CH₂)ₑ—R5 wherein d is 0 or 1; e is 0 to 6; X is O, NR⁶ or S(O)f where f is 0, 1 or 2; R⁵ is hydrogen, halogen, C₁₋₆ alkyl, C₂₋₆ alkyl, C₂₋₆ alkenyl, C₃₋₁₂ cycloalkyl, heterocyclyl, aryl, heteroaryl, hydroxyl, cyano, nitro, trihalomethyl, NR⁷R⁸, C₆H₄NR⁷R⁸, C₆H₄(CH₂)NR⁷R⁸, C(O)R⁷, C(O)NR⁷R⁸, OC(O)R⁷, OC(O)NR⁷R⁸, CO₂R⁷, OCO₂R⁷, SO₂R⁷, SO₂NR⁷R⁸, C(═NR⁷)NR⁷R⁸, NR⁷(C═NR⁷)NR⁷R⁸, NHC(O)R⁷ or N(C₁₋₃alkyl)C(O)R⁷; each R² is independently selected from hydrogen, cyano, halogen, trihalomethyl, OC₂₋₆alkyl, C₂₋₆alkyl, C₂₋₆alkenyl, C₂₋₆alkynyl, S(O)gC₁₋₆ alkyl where g is 0, 1 or 2, NC₁₋₆ alkyl(C₁₋₆ alkyl), hydroxyl or nitro; each R⁴ is independently selected from groups of the formula

—(Y)_{d}—(CH₂)ₑ—R³

wherein d is 0 or 1; e is 0 to 6; Y is O or S(O)_{f} where f is 0, 1 or 2; R³ is hydrogen, halogen, C₁₋₆ alkyl, C₂₋₆ alkyl, C₂₋₆ alkenyl, C₃₋₁₂ cycloalkyl, heterocyclyl, aryl, heteroaryl, hydroxyl, cyano, nitro, trihalomethyl, phthalamido, C₆H₄NR⁷R⁸, C₆H₄(CH₂)NR⁷R⁸, C(O)R⁷, C(O)NR⁷R⁸, OC(O)R⁷, OC(O)NR⁷R⁸, CO₂R⁷, OCO₂R⁷, SO₂R⁷, SO₂NR⁷R⁸ or C(═NR⁷)NR⁷R⁸; R⁶ is H, C₁₋₆ alkyl, C₂₋₆ alkyl, C₂₋₆ alkenyl, C₃₋₁₂ cycloalkyl, or heterocyclyl; R⁷ and R⁸ are each independently H, C₁₋₈ alkyl, C₂₋₆ alkenyl, SO₂C₁₋₆ alkyl, (CH₂)ₘ—C₃₋₁₂ cycloalkyl, (CH₂)ₘ-aryl, (CH₂)ₘ-heterocyclyl, (CH₂)ₘ-heteroaryl, wherein m=0, 1 or 2, or may, together with the nitrogen atom to which they are bound, form a heterocyclyl group; and wherein any of said alkyl, alkenyl and alkynyl groups may be optionally substituted with up to three members selected from halogen, hydroxyl, oxo, cyano, NR⁷R⁸, C₁₋₆ alkyl, OC₁₋₆ alkyl, S(O)C₁₋₆ alkyl, S(O)₂C₁₋₆ alkyl and SO₂NNR⁷R⁸; and

wherein any of said cycloalkyl, heterocyclyl, aryl, and heteroaryl groups may be optionally substituted with substituents selected from a group consisting of C₁₋₆ alkyl, C₁₋₆ alkoxy, C₁₋₆ alkylsulfenyl, C₁₋₆ alkylsulfinyl, C₁₋₆ alkylsulfonyl, hydroxy, oxo, mercapto, nitro, cyano, halogen, C₁₋₆ perfluoroalkyl, amino optionally substituted by C₁₋₆ alkyl, carbamoyl optionally substituted by C₁₋₆ alkyl, NR⁷R⁸, carboxy and aminosulfonyl optionally substituted by C₁₋₆ alkyl; with the proviso that only those substituted pyrazoles that are specific inhibitors of erbB4 are encompassed by the present invention.

In one embodiment, the small molecule agent used in the methods and compositions described herein is a member of the class of 3-aza-1-oxa-dibenzo[e,h]azulenes set forth in formula V:

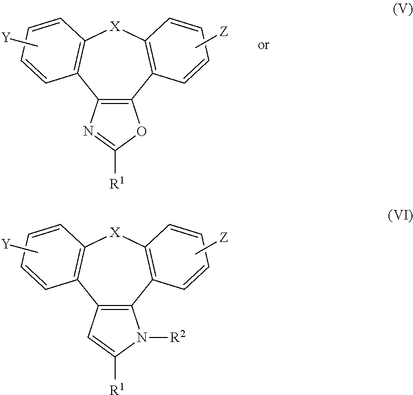

wherein X is O, S, S(═O), S(═O)₂, or NR^{a}, wherein R^{a} is hydrogen or a substituent selected from C₁-C₃-alkyl, C₁-C₃-alkanoyl, C₁-C₇-alkoxycarbonyl, C₇-C₁₀-arylalkyloxycarbonyl, C₇-C₁₀-aroyl, C₇-C₁₀-arylalkyl, C₃-C₇-alkylsilyl or C₅-C₁₀-alkylsilylalkyloxyalkyl;

Y and Z independently from each other mean one or more identical or different substituents linked to any available carbon atom selected from the group consisting of hydrogen; halogen, C₁-C₄-alkyl, C₂-C₄-alkenyl, C₂-C₄-alkinyl, halo-C₁-C₄ alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, thiol, C₁-C₄ alkylthio, sulfonyl, C₁-C₄ alkylsulfonyl, sulfinyl, C₁-C₄ alkylsulfinyl, carboxy, C₁-C₄ alkoxycarbonyl, cyano and nitro;

R¹ is hydrogen, CHO, (CH₂)₂COOH, (CH₂)₂CO₂CH₂CH₃, (CH₂)ₘL wherein m is an integer from 1 to 3, n is an integer between 0-3 and L is OH or Br, or a substituent set forth by formula VII:

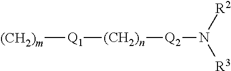

R² and R³ are either or both hydrogen, C₁-C₄-alkyl, aryl such as an aromatic ring or fused aromatic rings containing one ring of at least 6 carbon atoms or two rings with a total of 10 carbon atoms and conjugated double bonds; or together with N are heterocyclic having 5, or 6-member rings or heteroaryl fully saturated or partly unsaturated heterocycle group containing at least one hetero atom such as O, S or N and where said heterocycle is optionally substituted with one or two substituents such as halogen, C₁-C₄-alkyl, cyano, nitro, hydroxy, C₁-C₄-alkoxy, thiol, C₁-C₄-alkylthio, amino, N—(C₁-C₄) alkylamino, N,N-di(C₁-C₄-alkyl)amino, sulfonyl, C₁-C₄ alkylsulfonyl, sulfinyl, C₁-C₄ alkylsulfinyl; and wherein heteroaryl relates to aromatic and partially aromatic groups of a monocyclic or bicyclic ring with 4 to 12 carbon atoms and at least one of O, S or N and where said heteroaryl is optionally substituted with one or two substituents which are selected from halogen, C₁-C₄-alkyl, cyano, nitro, hydroxy, C₁-C₄-alkoxy, thiol, C₁-C₄-alkylthio, amino, N—(C₁-C₄)alkylamino, N,N-di(C₁-C₄-alkyl)amino, sulfonyl, C₁-C₄ alkylsulfonyl, sulfinyl, C₁-C₄ alkylsulfinyl;

In one embodiment, N in formula VI is substituted with a heterocycle or heteroaryl that is morpholine-4-yl, piperidine-1-yl, pyrrolidine-1-yl, imidazole-1-yl or piperazine-1-yl

Q₁ and Q₂ are either or both oxygen, CH₂, sulfur or a group set forth by:

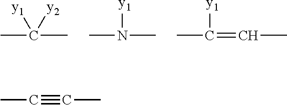

wherein y₁ and y₂ are either or both; hydrogen, halogen (F, Cl or Br); C₁-C₄-alkyl, aryl wherein aryl has the meaning as defined above, —OH, C₁-C₄-alkyloxy, C₁-C₄ alkanoyl, thiol, C₁-C₄-alkylthio, sulfonyl, C₁-C₄ alkylsulfonyl, sulfinyl, C₁-C₄ alkylsulfinyl, cyano, nitro or together form carbonyl or imino group, and of their pharmaceutically acceptable salts and solvates for the manufacture of pharmaceutical formulations for the treatment and prevention of diseases, damages and disorders of the central nervous system caused by disorders of neurochemical equilibrium of biogenic amines or other neurotransmitters.

In another embodiment, the compound of formula V used as the small molecule agent in the methods and compositions described herein is 1-oxa-8-thia-3-aza-dibenzo[e,h]azulene; 1,8-dioxa-3-aza-dibenzo[e,h]azulene; 3-(1-oxa-8-thia-3-aza-dibenzo[e,h]azulen-2-yl)-propionic acid ethyl ester; 3-(1,8-dioxa-3-aza-dibenzo[e,h]azulen-2-yl)-propionic acid ethyl ester; 2-methyl-1-oxa-8-thia-3-aza-dibenzo[e,h]azulene; 2-methyl-1,8-dioxa-3-aza-dibenzo[e,h]azulene; 11-chloro-2-methyl-1-oxa-8-thia-3-aza-dibenzo[e,h]azulene; 5-chloro-2-methyl-1-oxa-8-thia-3-aza-dibenzo[e,h]azulene; 11-chloro-2-methyl-1,8-dioxa-3-aza-dibenzo[e,h]azulene; 5-chloro-2-methyl-1,8-dioxa-3-aza-dibenzo[e,h]azulene; 1-oxa-8-thia-3-aza-dibenzo[e,h]azulene-2-carbaldehyde; 3-(1-oxa-8-thia-3-aza-dibenzo[e,h]azulen-2-yl)-propionic acid; 3-(1,8-dioxa-3-aza-dibenzo[e,h]azulen-2-yl)-propionic acid; (1-oxa-8-thia-3-aza-dibenzo[e,h]azulen-2-yl)-methanol; 3-(1-oxa-8-thia-3-aza-dibenzo[e,h]azulen-2-yl)-propane-1-ol; 3-(1,8-dioxa-3-aza-dibenzo[e,h]azulen-2-yl)-propane-1-ol; 2-bromomethyl-1-oxa-8-thia-3-aza-dibenzo[e,h]azulene; 2-bromomethyl-1,8-dioxa-3-aza-dibenzo[e,h]azulene; 2-bromomethyl-5-chloro-1-oxa-8-thia-3-aza-dibenzo[e,h]azulene; 2-bromomethyl-11-chloro-1-oxa-8-thia-3-aza-dibenzo[e,h]azulene; 2-bromomethyl-5-chloro-1,8-dioxa-3-aza-dibenzo[e,h]azulene; 2-bromomethyl-11-chloro-1,8-dioxa-3-aza-dibenzo [e,h]azulene; dimethyl-[2-(1-oxa-8-thia-3-aza-dibenzo[e,h]azulen-2-ylmethoxy)-ethyl]-am-ine; dimethyl-[3-(1-oxa-8-thia-3-aza-dibenzo[e,h]azulen-2-ylmethoxy)-propyl]-a-mine; dimethyl-{2-[3-(1-oxa-8-thia-3-aza-dibenzo[e,h]azulen-2-yl)-propoxy]-ethy-11-amine; dimethyl-{3-[3-(1-oxa-8-thia-3-aza-dibenzo[e,h]azulen-2-yl)-propoxy]-prop-yl}-amine; {2-[3-(1,8-dioxa-3-aza-dibenzo[e,h]azulen-2-yl)-propoxy]-ethyl}-dimethyla-mine; {3-[3-(1,8-dioxa-3-aza-dibenzo[e,h]azulen-2-yl)-propoxy]-propyl}-dimethyl-amine; [2-(1,8-dioxa-3-aza-dibenzo[e,h]azulen-2-ylmethoxy)-ethyl]-dimethylamine; [3-(1,8-dioxa-3-aza-dibenzo[e,h]azulen-2-ylmethoxy)-propyl]-dimet-hylamine; 2-(5-chloro-1-oxa-8-thia-3-aza-dibenzo[e,h]azulen-2-ylmethoxy)-ethyl]-dim-ethylamine; [3-(5-chloro-1-oxa-8-thia-3-aza-dibenzo[e,h]azulen-2-ylmethoxy)-propyl]-d-imethylamine; [2-(11-chloro-1-oxa-8-thia-3-aza-dibenzo[e,h]azulen-2-ylmethoxy)-ethyl]-d-imethylamine; [3-(11-chloro-1-oxa-8-thia-3-aza-dibenzo[e,h]azulen-2-ylmethoxy)-propyl]-dimethylamine; [2-(5-chloro-1,8-dioxa-3-aza-dibenzo[e,h]azulen-2-ylmethoxy)-ethyl]-dimet-hylamine; [3-(5-chloro-1,8-dioxa-3-aza-dibenzo[e,h]azulen-2-ylmethoxy)-propyl]-dime-thylamine; [2-(11-chloro-1,8-dioxa-3-aza-dibenzo[e,h]azulen-2-ylmethoxy)-ethyl]-dime-thylamine; or [3-(11-chloro-1,8-dioxa-3-aza-dibenzo[e,h]azulen-2-ylmethoxy)-propyl]-dim-ethylamine.

In one embodiment, the compound of formula VI used as the small molecule agent in the methods and compositions described herein is

In one embodiment, the agent administered to the subject in an amount sufficient to inhibit the attachment of erbB4, erbB3 or their combination to NRG1 is an small molecule agent, specific against erbB4, erbB3 or their combination, or its fraction. In one embodiment, the antibodies used in the methods described herein is produced by using the subject's own isolated erbB4, erbB3 or their combination to generate the small molecule agent.

In one embodiment, the term “small molecule agent” include complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include small molecule agent constant domains. In another embodiment, F(ab)'s lack constant domains which are required for complement fixation. scFvs are composed of an small molecule agent variable light chain (V_{L}) linked to a variable heavy chain (V_{H}) by a flexible linker. scFvs are able to bind antigen and can be rapidly produced in bacteria. The invention includes antibodies and small molecule agent fragments which are produced in bacteria and in mammalian cell culture. An small molecule agent obtained from a bacteriophage library can be a complete small molecule agent or an small molecule agent fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies described herein can be monoclonal antibodies (Mab) in one embodiment, or polyclonal antibodies in another embodiment. Antibodies of the invention which are useful for the compositions and methods described herein can be from any source, and in addition may be chimeric. In one embodiment, sources of antibodies can be from a mouse, or a rat, or a human in other discrete embodiments. Antibodies of the invention which are useful for the compositions and methods of the invention have reduced antigenicity in humans, and in another embodiment, are not antigenic in humans. Chimeric antibodies as described herein contain in one embodiment, human amino acid sequences and include humanized antibodies which are non-human antibodies substituted with sequences of human origin to reduce or eliminate immunogenicity, but which retain the binding characteristics of the non-human small molecule agent. In another embodiment, the small molecule agent capable of inhibiting the attachment of erbB4 to NRG1, is the erbB4 small molecule agent SC-348.

In one embodiment, schizophrenia is associated with NMDAR hypofunction. In another embodiment, cross-talk between NRG1-erbB4 signaling and NMDAR function are implicated in schizophrenia. In another embodiment, erbB4 signaling is enhanced in schizophrenia and NRG1 stimulation can further mediate NMDAR hypofunction. In one embodiment, erbB4-mediated suppression of NMDAR signaling is an important mechanism underlying the susceptibility to NMDAR hypofunction in schizophrenia and the methods and compositions described herein may be used in certain embodiments to treat the pathology by extrinsic stimulation of NMDAR.

N-Methyl-D-aspartate receptors (NMDARs) are a subtype of ionotropic glutamate receptors (iGluRs) that serve critical functions in physiological and pathological processes in the nervous system, including neuronal development, plasticity and neurodegeneration. NR1 (a glycine receptor) and NR2A-D subunits (Glutamate receptors) co-assemble to form conventional NMDARs whose activation requires glycine and glutamate as co-agonists. In one embodiment, stimulating NMDAR function is done by contacting the NMDAR with Glycine, Glutamate, (2S,2′R,3′R)-2-(2′, 3′-dicarboxycyclopropyl)glycine (DCG-IV), (2S, 1′S,2′S)-2-(carboxycyclopropyl)-glycine (L-CCG-I), Dopamine D₁ receptor agonist SKF81297 NMDA or their combination. In one embodiment, compounds capable of stimulating NMDAR are used in the methods described herein as a separate and distinct step, or in one embodiment as a treatment modality carried out before, during or following the administration of the antibodies described herein.

In one embodiment, provided herein is method of treating a neuropsychiatric disorder in a subject, comprising the step of administering to the subject an small molecule agent specific against erbB4, erbB3 or their combination, or fragments thereof in an amount sufficient to inhibit the attachment of erbB4, erbB3 or their combination to NRG1, PSD-95 or both, and further subjecting the subject to at least one other treatment modality, in one embodiment prior to, or during or after the administration of the an small molecule agent specific against erbB4, erbB3 or their combination, or fragments thereof in other discrete embodiments. In one embodiment, the additional teatment modality is a pharmacological therapy comprising administration of agents capable of stimulating NMDAR in certain embodiments. In one embodiment, is electro-shock therapy, or surgery in another embodiment, or their combination.

As will be understood by those skilled in the art, the immunologically binding reagents encompassed by the term “antibodies or their fragment” extend in certain embodiments, to all antibodies from all species including dimeric, trimeric and multimeric antibodies; bispecific antibodies; chimeric antibodies; human and humanized antibodies; recombinant and engineered antibodies, and fragments thereof. The term “antibodies or their fragment” refers in another embodiment to any antibody-like molecule that has an antigen binding region, and this term includes small molecule agent fragments such as Fab′, Fab, F(ab′)₂, single domain antibodies (DABs), Fv, scFv (single chain Fv), linear antibodies, diabodies, and the like. The techniques for preparing and using various antibody-based constructs and fragments are well known in the art In one embodiment, the anti-erbB4, or erbB3 fragment used in the methods and compositions described herein, is Fc, or Fab, F(ab′), F(ab′)₂ or a combination thereof in other embodiments. In another embodiment, the anti-erbB4, or erbB3 fragment used in the methods and compositions described herein, is Fc, or Fab, F(ab′), F(ab′)₂ or a combination thereof in other embodiments.

The term “antibody fragment” also includes any synthetic or genetically engineered protein that acts like an small molecule agent by binding to a specific antigen to form a complex. In one embodiment, antibody fragments include isolated fragments, “Fv” fragments, consisting of the variable regions of the heavy and light chains, recombinant single chain polypeptide molecules in which light and heavy chain variable regions are connected by a peptide linker (“sFv proteins”), and minimal recognition units consisting of the amino acid residues that mimic the hypervariable region. In one embodiment, the antibody is a variable regions of the heavy and light chains, or recombinant single chain polypeptide molecules in which light and heavy chain variable regions are connected by a peptide linker (“sFv proteins”), and minimal recognition units consisting of the amino acid residues that mimic the hypervariable region in other embodiments.

In one embodiment, provided herein is a method of treating, inhibiting or suppressing, or ameliorating symptoms associated with a neuropsychiatric disorder in a subject, inhibiting or suppressing a neuropsychiatric disorder in a subject, or ameliorating symptoms associated with a neuropsychiatric disorder in a subject, comprising the step of administering to the subject a small molecule agent, or an antibody or its fragment in an amount sufficient to inhibit the attachment of erbB4, erbB3 or their combination to NRG1.

In another embodiment, provided herein is a composition for treating a neuropsychiatric disorder in a subject comprising a small molecule agent, or an antibody or its fragment and a pharmaceutically acceptable carrier, excipient, flow agent, processing aid, diluent or a combination thereof, thereby inhibiting the attachment of erbB4, erbB3 or their combination to NRG1.

In one embodiment, provided herein is a method of inhibiting neuregulinl (NRG1) activation of protein kinase B (AKT), extracellular signal-regulated kinase (ERK) or both, comprising the step of contacting a brain cell with a composition comprising a small molecule agent capable of inhibiting the attachement of erbB4 to NRG1.

In another embodiment, provided herein is a method of screening for an agent for the treatment of neuropsychiatric disorders, comprsing the steps of obtaining a biological sample from a subject or pool of subjects diagnosed with having a neuropsychiatric disorder; incubating the biological sample with a candidate agent for a predetermined period; following incubation, contacting the biological sample with neuregulinl; and analyzing whether the candidate agent is capable of inhibiting the attachement of erbB4 to NRG1, wherein a candidate agent capable of inhibiting the attachement of erbB4 to NRG1 is useful for the treatment of psychiatric disorder.

In one embodiment, provided herein is a method of inhibiting NMDAR 2B phosphorylation in a cell, comprising the step of contacting the cell with a composition comprising an agent capable of inhibiting erbB4-NRG1 attachment.

In another embodiment, provided herein is a method of inhibiting NMDAR1 association with phosphatidyl inositole phospholipase C-γ (PIPLCγ) in a cell, comprising the step of contacting the cell with a small molecule agent capable of inhibiting erbB4-NRG1 attachment.

In one embodiment, the agent capable of inhibiting NRG1-erbB4 attachement is AG1487, CI1103, AG879, PD158780, PD168393, GSK1495829A, GSK1521232A, or their combination.

In one embodiment, provided herein is a method of ameliorating symptoms associated with a neuropsychiatric disorder in a subject, comprising contacting the subject via implant administration with AG1487 in an amount sufficient to inhibit the attachment of erbB4, erbB3 or their combination to NRG1, thereby modulating synaptic plasticity.

In another embodiment, provided herein is a method of ameliorating symptoms associated with a neuropsychiatric disorder in a subject, comprising contacting the subject via implant administration with GSK1495829A in an amount sufficient to inhibit the attachment of erbB4, erbB3 or their combination to NRG1, thereby modulating synaptic plasticity.

In one embodiment, the compositions described herein comprise one or more additional active pharmaceutical agent for the treatment of psychiatric disorders. In another embodiment, the one or more additional agent will be administered as an integral part of the compositions described herein, or in another embodiment, before, simultaneously with or after the administration of the compositions described herein.

Nrg1-ErbB4 signaling plays an important role in synaptic plasticity, in one embodiment, by regulating the levels of pre- and postsynaptic receptors and ion channels.

In one embodiment, contacting the subject with the agents described herein, shows decreased release of phosphorylated extracellular signal-regulated kinase (p-Erk). In another embodiment, phosphorylated extracellular signal-regulated kinase (p-Erk) is a marker of stress.

In one embodiment, the compsitions described herein, used in the methods described herein is administered intravenously, intracavitarily, subcutaneously, intratumoraly, or a combination thereof.

“Intracavitary administration”, as used herein, refers to administering a substance directly into a body cavity of a mammal. Such body cavities include the peritoneal cavity, the pleural cavity and cavities within the central nervous system, including the orbit of the eye.

In another embodiment of this invention, the small molecule agent described herein, or antibodies and their fragment is administered via the subcutaneous route. According to the present invention the antibodies described herein may be administered as a pharmaceutical composition containing a pharmaceutically acceptable carrier. The carrier must be physiologically tolerable and must be compatible with the active ingredient. Suitable carriers include, sterile water, saline, dextrose, glycerol and the like. In addition, the compositions may contain minor amounts of stabilizing or pH buffering agents and the like. The compositions are conventionally administered through parenteral routes, with intravenous, intracavitary or subcutaneous injection being preferred.

The preparation comprising the small molecule agent, polyclonal antibodies described herein, or their fragments; are administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

In one embodiment, the small molecule agent used in the compositions and methods described herein is α-Cyano-(3,5-di-t-butyl-4-hydroxy)thiocinnamide, (AG 879), or in another embodiment, an small molecule agent to the ‘a’-tail cytoplasmic domain of NRG1 (Neuregulin-1a/b1/2(C-20) (SC-348). In one embodiment, the small molecule agent needs to be in contact with culture cells for about 60 min for its optimal inhibitory effects.

In one embodiment, ErbB-4 is a transmembrane receptor tyrosine kinase that regulates cell proliferation and differentiation. After binding of its ligand heregulin (HRG) or activation of protein kinase C (PKC) by 12-O-tetradecanoylphorbol-13-acetate (TPA), the ErbB-4 ectodomain is cleaved by a metalloprotease. A subsequent cleavage by gamma-secretase releases the ErbB-4 intracellular domain from the membrane and facilitates its translocation to the nucleus. In another embodiment gamma-Secretase cleavage was prevented by chemical inhibitors or a dominant negative presenilin In one embodiment, gamma-Secretase cleavage of ErbB-4 represents another mechanism for receptor tyrosine kinase-mediated signaling and its inhibition will affect erbB4 signaling pathways. Accordingly in one embodiment, the methods described herein for the treatment of neuro-psychiatricdisorders, further comprise administering to the subject the compositions described herein, as well as γ-secretase inhibitors.

Protein kinase C (PKC) exists as a family of closely related subspecies, having a diverse distribution in brain, with high levels in presynaptic nerve terminals, and, together with other kinases, in one embodiment, play a crucial role in the regulation of synaptic plasticity. In one embodiment, PKC is a major intracellular mediator of signals generated upon external stimulation of cells via a variety of neurotransmitter receptors such as muscarinic M1, M3, M5 receptors in one embodiment, or, noradrenergic α1 receptors, metabotropic glutamatergic receptors such as NMDAR, or serotonergic 5-HT_{2A} receptors and their combination in other embodiments.

In one embodiment, particulate (membrane) PKC is a more active form of PKC. In another embodiment PKC activity and PKC translocation in response to serotonin in platelets obtained from Bipolar Disorder subjects before and during lithium treatment, show that the ratios of platelet membrane-bound to cytosolic PKC activities are elevated in the manic subjects. In another embodiment, serotonin-elicited platelet PKC translocation is enhanced in subjects in the manic stage. In another embodiment, measured PKC isozyme levels, activity and translocation in post-mortem brain tissue from BD patients show increased PKC activity and translocation in BD brains compared to controls, effects which were accompanied by elevated levels of selected PKC isozymes in cortices of BD subjects. In view of the involvement of the PKC signaling system in the pathophysiology of BD, in another embodiment, PKC inhibitors are very useful agents in the treatment of mania.

In one embodiment, using the methods and compositions described herein, PKC activation is attenuated, and is thereby useful in the treatment of bipolar disorder. Accordingly, provided herein is a method of treating, or ameliorating symptoms associated with Bipolar Disorder, comprising the step of administering to a subject diagnosed with BD with a composition comprising an agent capable of inhibiting the attachment of erbB4, to NRG1, PSD-95 or both. This agent is GSK1495829A in one embodiment, or AG1487, CI1103, AG879, PD158780, PD168393, GSK1521232A or their combination in other discrete embodiments. In one embodiment, the compositions further comprise lithium.

In another embodiment, the methods described herein are carried out using the compositions described herein. Accordingly and n one embodiment, provided herein is a composition for treating a neuropsychiatric disorder in a subject comprising a small molecule agent specific against erbB4, erbB3 or their combination, or fragments thereof in an amount sufficient to inhibit the attachment of erbB4, erbB3 or their combination to NRG1, and a pharmaceutically acceptable carrier, excipient, flow agent, processing aid, diluent or a combination thereof.

Biologically active derivatives or analogs of the proteins described herein include in one embodiment peptide mimetics. Peptide mimetics can be designed and produced by techniques known to those of skill in the art. (see e.g., U.S. Pat. Nos. 4,612,132; 5,643,873 and 5,654,276, the teachings of which are incorporated herein by reference). These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In one embodiment, the composition further comprises a carrier, excipient, lubricant, flow aid, processing aid or diluent, wherein said carrier, excipient, lubricant, flow aid, processing aid or diluent is a gμM, starch, a sugar, a cellulosic material, an acrylate, calciμM carbonate, magnesiμM oxide, talc, lactose monohydrate, magnesiμM stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fμMaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesiμM stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleμM, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient. An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the term “administering” or “contacting” refers to bringing a subject in contact with the compositions provided herein. For example, in one embodiment, the compositions provided herein are suitable for oral administration, whereby bringing the subject in contact with the composition comprises ingesting the compositions. A person skilled in the art would readily recognize that the methods of bringing the subject in contact with the compositions provided herein, will depend on many variables such as, without any intention to limit the modes of administration; the cardiovascular disorder treated, age, pre-existing conditions, other agents administered to the subject, the severity of symptoms, location of the affected area and the like. In one embodiment, provided herein are embodiments of methods for administering the compounds of the present invention to a subject, through any appropriate route, as will be appreciated by one skilled in the art. In one embodiment, provided herein is a method of inhibiting or suppressing a neuropsychiatric disorder, such as schizophrenia, comprising the step of administering GSK1521232A, or GSK1495829A in an amount sufficient to inhibit the attachment of erbB4, erbB3 or their combination to NRG1, thereby modulating synaptic plasticity, wherein the administration is in a formulation suitabble for oral administration and in another embodiment, further comprises excipients, protease inhibitors, and the like.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gμM tragacanth, acacia, cornstarch, or gelatin; excipients, such as dicalciμM phosphate; a disintegrating agent, such as corn starch, potato starch, alginic acid and the like; a lubricant, such as magnesiμM stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pμMp, a transdermal patch, liposomes, or other modes of administration. In one embodiment, a pμMp may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albμMin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodiμM metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials. In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Example 1

### Agents Inhibit Contact of erbB4 and NRG1 in Postmortem Brain of Schizophrenia Patients

The main goal of this Example was to test a small molecule compound that is a specific erbB4 inhibitor recently developed, for its abilities to inhibit NRG1-erbB4 activation and to modulate NMDAR signalling in the brains of schizophrenia patients. To that end, it was proposed to test this compound in the post-mortem brains of SCZ subjects. The objectives set was to establish the experimental paradigm, combining pharmacologic inhibition experiment with the stimulation paradigm in the post-mortem tissues. In addition, the experimental paradigm was extended to an in vitro neuroepithelial system, olfactory epithelial cultures.

For an in vitro system, human olfactory epithelial (hOE) cultures were chosen, developed by applicant. These neuroepithelial culture are thought to contain some of molecular and cellular characteristics of an individual. It is an important addition to test the effects of the compound in olfactory epithelial cultures derived from patients with schizophrenia patients.

To test hOE cultures for NRG1 signalling and its inhibition, commercially available erbB inhibitors, AG 1478 and AG 879 were used. hOE culture cells derived from a healthy subject, 43 YO AA male, were grown to confluence and were deprived of serμM for 4 hours. Culture cells were then incubated with AG 1478 or AG 879, which were then tested for downstream signalling, ERK, AKT and GSK-3. As an internal control experiment, the cells were also incubated with inhibitors for ERK, AKT or trkb activation, U0126, wortmannin or K-252 respectively.

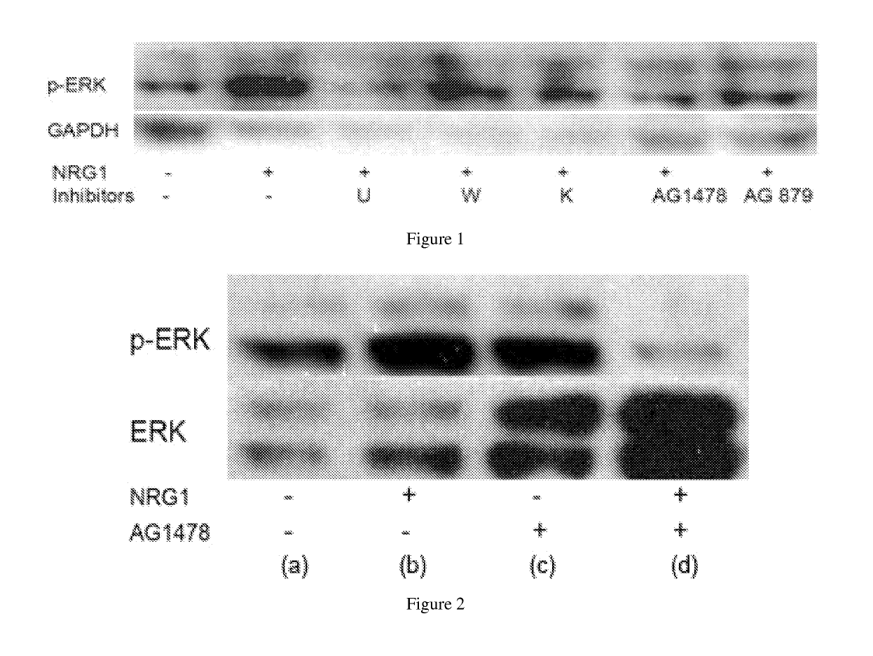

FIG. 1 is a representative western blot demonstrating that NRG1 enhances the ratios of phosphorylated ERK (p-ERK) with respect to GAPDH significantly. These, however, were attenuated by AG 1478 or AG 879. As expected, NRG1 induced p-ERK activation was significantly reduced in the presence of U0126 (MEK inhibitor). In the presence of wortmannin (AKT inhibitor) or K-252, ERK activation was also reduced but to a lesser degree. Our results show that K-252, known as trkb receptors, also affects erbB receptors to a degree.

So far, durations of the incubation with inhibitors were varied. The twenty min duration appears to be as effective as up to 60 min incubation for these inhibitors. Two other erbB inhibitors, PD 158780 and PD168393, are tested in various doses and durations. Once the paradigm is established, it is possible to test the effects of inhibitors in OE cultures derived from SCZ patients compared with those from matched controls (of healthy individual).

erbB stimulation/inhibition experiment wastested in post-mortem brain tissues. FIG. 2 represents an experiment, in which the PFC of a healthy control subject was stimulated with NRG1 in the presence of AG1478. In this experiment, the tissues were incubated with the inhibitor for 15 min before added with 100 ng/ml of NRG1. When the post-mortem PFC was stimulated with NRG1, ERK activation, assessed by the ratios of p-ERK with respect to ERK, was clearly enhanced. When the tissues were pre-incubated with the inhibitor, however, the NRG1 induced ERK activation was significantly reduced.

PD15780 and PD168393 are tested to construct the dose response curves and to identify the optimal duration for the inhibitors efficiencies.

### Example 2

### GSK1495829A inhibits NRG1 Induced Signalling in Human Olfactory Neuro-Epithelial (hOE) Cultures in a Dose Dependent Manner

To test the potency and the dose-response relationships for GSK1495829A, first hOE culture cells were employed. hOE culture cells derived from a healthy subject, 43 year old African-American male, were grown to confluence and were deprived of serum for 4 hours. Culture cells were then incubated with varied concentrations of GSK1495829A and GSK1521232A for 15 min and subsequently added with 100 ng/ml of NRG1. Tissue extracts were then analyzed by immunoblotting for the downstream signalling mechanisms, ERK and AKT.

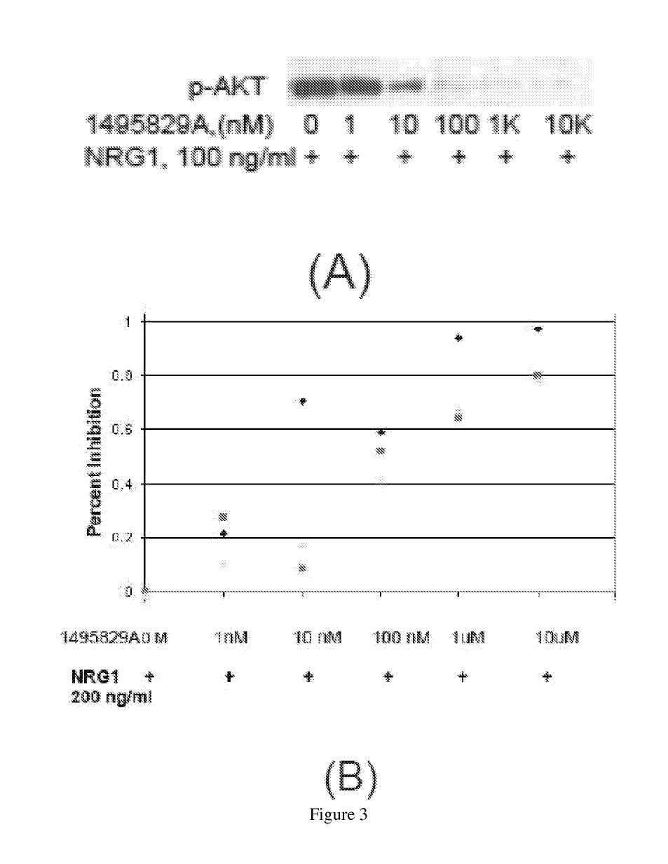

As shown in FIG. 3A, a representative blot, GSK149829A inhibited NRG1 induced activation of AKT in a dose dependent manner. A similar dose response curve was also shown for the activation of both AKT or ERK (FIG. 3B).

GSK1521232A appeared to differ from GSK1495829A in its capacity to inhibit NRG1 signaling. In three separate experiments, GSK1521232A inconsistently inhibited the activation of AKT and ERK except at the concentration of 10 μM (data not shown).

### Example 3

### GSK1495829A Inhibits NRG1 Induced Signalling in Postmortem Human Prefrontal Cortex Tissues in a Dose Dependent Manner

GSK1495829A was tested in post-mortem mouse and human brain tissues. FIG. 4 represents an experiment, in which the PFC tissues of a healthy control subject were pre-incubated with varied concentrations of the inhibitor for 15 min before the ligand stimulation with 100 ng/ml of NRG1. While GSK1495829A clearly inhibited ERK activation in post-mortem brain tissues, the extent to which NRG1 induced ERK activation was reduced was not as robust as in OE culture cells. One possible explanation could be that the brain tissues are less permeable to the inhibitors than OE culture cells.

To enhance the permeability of the inhibitors, tissue slices were pre-incubated with the inhibitors for 60 min before added with NRG1. In any event the 10 μM concentration of GSK14958292A did not reduce the activation of ERK or AKT, nor did AG1467 (FIG. 5). It is also of note that NRG1 stimulation, followed by 60 min pre-incubation with the vehicle, failed to enhance the activation of ERK. Thus, it is highly likely that the lack of inhibitory efficacy of GSK14958292A was because the extended pre-incubation period does not support the viability of the signalling mechanisms in the post-mortem brain stimulation paradigm.

In sum, the experiments disclosed, clearly demonstrated that GSK1495829A inhibits NRG1 induced activation of intracellular signalling in hOE culture cells as well as in human post-mortem brain tissues in a dose dependent manner. The inhibitory effects of GSK1521232A appear to be less clear cut.

### Example 4

### GSK1495829A and GSK1521232A Inhibit NRG1 Induced Signalling in Human Olfactory Neuro-Epithelial (hOE) Cultures in a Dose Dependent Manner

hOE culture cells derived from a healthy subject, 27 year old Caucasian female, were grown to confluence and were deprived of serum for 4 hours. Culture cells were then incubated with varied concentrations of GSK1495829A or with GSK1521232A for 15 min and subsequently added with 100 ng/ml of NRG1. Tissue extracts were then analyzed by immunoblotting for the downstream signalling mechanisms, ERK and AKT.

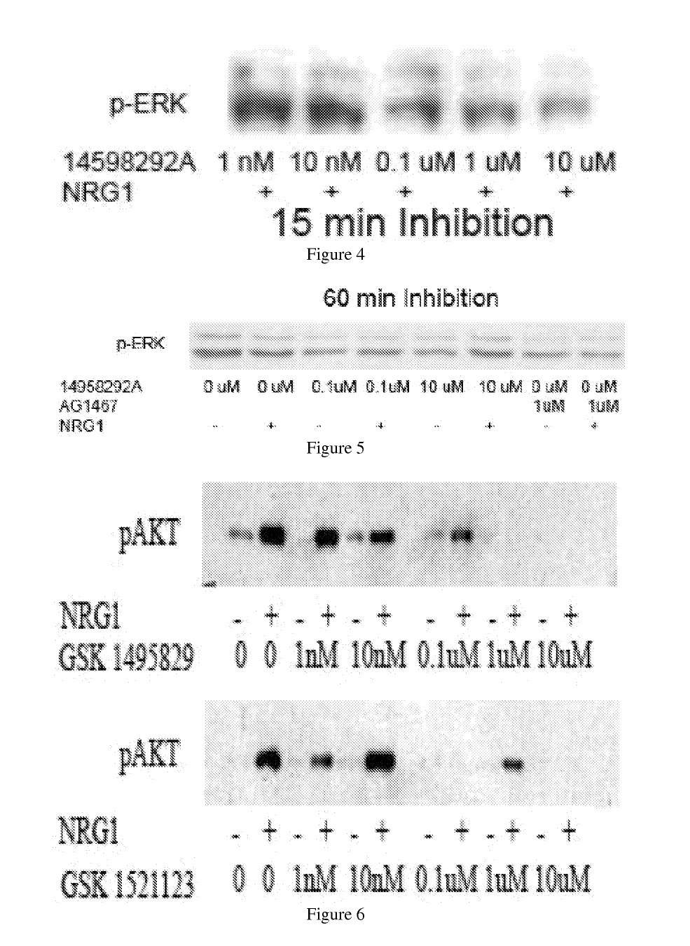

As shown in FIG. 6, a representative blot, GSK149829A inhibited NRG1 induced activation of AKT in a dose dependent manner. Such inhibition was also shown for the activation of ERK (FIG. 6B). GSK1521232A appeared to inhibit NRG1 signalling yet somewhat inconsistently.

### Example 5

### NRG1 Attenuates NMDA Induced Phosphorylation of pY1472 of NMDAR 2B

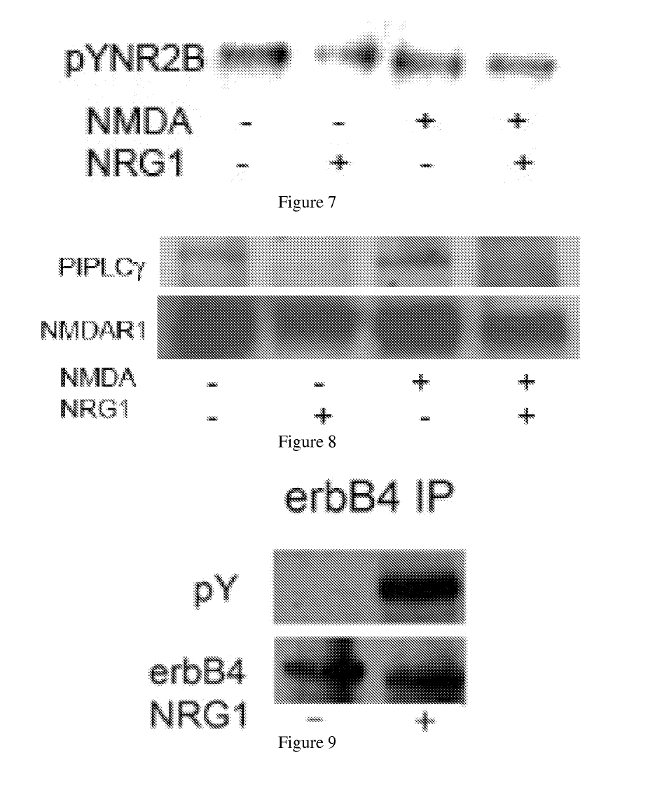

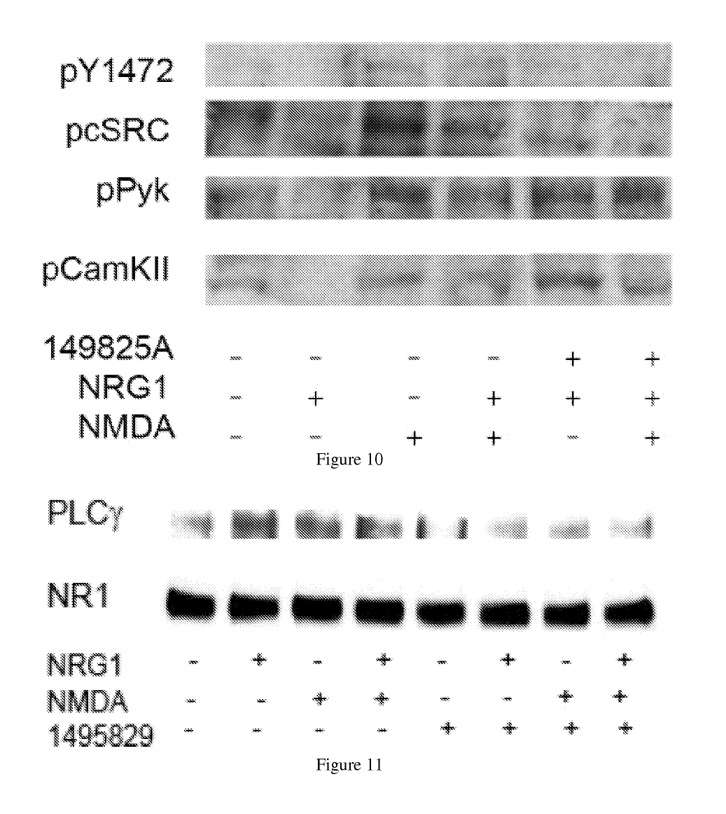

To test whether the two GSK compounds attenuate NRG1-induced modulation of NMDA receptor signaling in hOE cultures, hOE cultures were incubated with 100 ng/ml of NRG1 and 100 μM of NMDA+1 μM of glycine, in the presence or absence of 1 μM of GSK1495829A. Phosphorylation of 1472 tyrosine residues of NMDAR2B and the association of NMDAR1 with PIPLCγ for measures of the activation of NMDAR. FIG. 7 shows that pYNMDAR2B is attenuated in the presence of NRG1, which is in parallel to what was observed in brain tissues. Considering that hOE cultures do not express PSD-95, unlike brain tissues, this shows that NRG1 induced modulation of pYNMDAR2B is not mediated by PSD-95

### Example 6

### NRG1 Induced Modulation of NMDA Receptor Signalling in Mouse Brain Tissues

To examine if compound X attenuates NRG1 induced modulation of NMDA receptor signaling, post-mortem mouse brain tissue slices were incubated with 100 ng/ml of NRG1 and 100 μM of NMDA+1 μM of glycine, in the presence and absence of 1 μM of GSK1495829A. FIG. 8 shows that NRG1 decreases the association of NMDAR1 with PIPLCγ.

### Example 7

### Modulation of erbB4 Activation by GSK1495829A

As a direct test of GSK1495829A's inhibition of erbB4, OVCAR-3 cells, human ovarian carcinoma cell line, expressing erbB4 abundantly, were grown to confluence and incubated with NRG1 in the presence and absence of GSK1495829A. Protein extracts were immuno-precipitated with antibodies for erbB4 and were probed for phosphotyrosine (pY). FIG. 9 shows that NRG1 stimulation dramatically enhances pY content of erbB4. GSK1495829A attenuates the NRG1 induced increases in the pY content of erbB4. The effects of GSK1495829A on erbB4 activation was also tested in extracts of mouse and human brain tissues and the results are currently being processed.

### Example 8

### Modulation of NMDA Receptor Signalling in Neural Cells by GSK1495829A

BE(2)-M17 cells, a human neuroblastoma cell line, were grown in the presence of retinoic acid to about 75% confluence. BE cells were incubated with 200 ng/ml of NRG1 and 100 μM of NMDA+1 μM of glycine, in the presence and absence of 1 μM of GSK1495829A. NMDA stimulation enhanced pSRC, pPyK, pY1472, pCAMKII and pPyk in these cells. NMDA induced enhancement of pSRC, pPyK, pY1472 was attenuated in the presence of NRG1. In the presence of GSK1495829A, the overall activation of these molecules was attenuated.

### Example 9

### Modulation of NMDA Receptor Signalling by GSK1495829A

Human postmortem brain tissue slices were incubated with 200 ng/ml of NRG1 and 100 μM of NMDA+1 μM of glycine, in the presence and absence of 10 nM of GSK1495829A. Interestingly, 10 nM of GSK1495829A overall decreased the association of NR1 with PIPLCγ.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method of treating a neuropsychiatric disorder in a subject, comprising the step of administering to the subject a small molecule agent, in an amount sufficient to inhibit the attachment of erbB4, to NRG1, PSD-95 or both.

2. A method of inhibiting or suppressing a neuropsychiatric disorder in a subject, comprising the step of administering to the subject a small molecule agent in an amount sufficient to inhibit the attachment of erbB4 to NRG1.

3. A method of ameliorating symptoms associated with a neuropsychiatric disorder in a subject, comprising the step of administering to the subject a small molecule agent in an amount sufficient to inhibit the attachment of erbB4 to NRG1

4. The method of any one of claims 1, whereby the small molecule agent is AG1487, CI1103, AG879, PD158780, PD168393, GSK1495829A, GSK1521232A or their combination.

5. The method of any one of claims 1, whereby the small molecule agent is an antibody fragment specific against erbB4, wherein the fragment is Fc, Fab, F(ab′), F(ab′)₂ or a combination thereof.

6. The method of any one of claims 1, whereby the a small molecule agent is administered intravenously, intracavitarily, subcutaneously, intracranially, or a combination thereof.

7. The method of any one of claims 1, further comprising subjecting the subject to at least one other treatment modality, prior to, during or after the administration of the an small molecule agent specific against erbB4, or fragments thereof.

8. The method of claim 7, whereby the other treatment modality is electro-shock therapy, pharmacological therapy, surgery or a combination thereof.

9. The method of any one of claims 1, whereby the neuropsychiatric disorder is schizophrenia, bipolar disorder (BD), monopolar depression, anxiety, or obsessive-compulsive disorderor (OCD).

10. The method of any one of claims 1, further comprising administering to the subject γ-secretase inhibitor, in an amount effective to inhibit the releases of ErbB-4 intracellular domain from the membrane.

11. The method of any one of claims 1, further comprising administering to the subject wortmannin, lithium or both.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A method of inhibiting neuregulinl (NRG1) activation of protein kinase B (AKT), extracellular signal-regulated kinase (ERK) or both, comprising the step of contacting a brain cell with a composition comprising a small molecule agent capable of inhibiting the attachement of erbB4 to NRG1.

24. The method of claim 23, whereby the small molecule agent is AG1487, CI1103, AG879, PD158780, PD168393, GSK1495829A, GSK1521232A or their combination.

25. The method of claim 23, whereby the step of contacting is via intravenously, intracavitarily, subcutaneously, intracranially administration of the small molecule agent, or a combination thereof.

26. The method of claim 23, whereby the composition further comprises γ-secretase inhibitor, wortmanin, lithium or both.

27. A method of screening for an agent for the treatment of neuropsychiatric disorders, comprsing the steps of obtaining a biological sample from a subject or pool of subjects diagnosed with having a neuropsychiatric disorder; incubating the biological sample with a candidate agent for a predetermined period; following incubation, contacting the biological sample with neuregulin1; and analyzing whether the candidate agent is capable of inhibiting the attachement of erbB4 to NRG1, wherein a candidate agent capable of inhibiting the attachement of erbB4 to NRG1 is useful for the treatment of psychiatric disorder.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)
